SEQUENCE LISTING (7 sequences)

SEQ ID NO: 1 - DNA, 1059 bp, Homo sapiens
atggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 2 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - DNA, 1080 bp, Mus musculus
atggaaccgatcagtgtgagtatatacacttctgataactactctgaagaagtgggttctggagactatgactccaacaaggaaccctgcttccgggatgaaaacgtccatttcaataggatcttcctgcccaccatctacttcatcatcttcttgactggcatagtcggcaatggattggtgatcctggtcatgggttaccagaagaagctaaggagcatgacggacaagtaccggctgcacctgtcagtggctgacctcctctttgtcatcacactccccttctgggcagttgatgccatggctgactggtactttgggaaatttttgtgtaaggctgtccatatcatctacactgtcaacctctacagcagcgttctcatcctggccttcatcagcctggaccggtacctcgctattgtccacgccaccaacagtcagaggccaaggaaactgctggctgaaaaggcagtctatgtgggcgtctggatcccagccctcctcctgactatacctgacttcatctttgccgacgtcagccagggggacatcagtcagggggatgacaggtacatctgtgaccgcctttaccccgatagcctgtggatggtggtgtttcaattccagcatataatggtgggtctcgtcctgcccggcatcgtcatcctctcctgttactgcatcatcatctctaagctgtcacactccaagggccaccagaagcgcaaggccctcaagacgacagtcatcctcatcctagctttctttgcctgctggctgccatattatgtggggatcagcatcgactccttcatccttttgggggtcatcaagcaaggatgtgacttcgagagcatcgtgcacaagtggatctccatcacagaggccctcgccttcttccactgttgcctgaaccccatcctctatgccttcctcggggccaagttcaaaagctctgcccagcatgcactcaactccatgagcagaggctccagcctcaagatcctttccaaaggaaagcggggtggacactcttccgtctccacggagtcagaatcctccagttttcactccagctaa

SEQ ID NO: 4 - protein, 359 aa, Mus musculus
MEPISVSIYTSDNYSEEVGSGDYDSNKEPCFRDENVHFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVYVGVWIPALLLTIPDFIFADVSQGDISQGDDRYICDRLYPDSLWMVVFQFQHIMVGLVLPGIVILSCYCIIISKLSHSKGHQKRKALKTTVILILAFFACWLPYYVGISIDSFILLGVIKQGCDFESIVHKWISITEALAFFHCCLNPILYAFLGAKFKSSAQHALNSMSRGSSLKILSKGKRGGHSSVSTESESSSFHSS

SEQ ID NO: 5 - protein, 8 aa, Artificial Sequence
  Ovalbumin-derived peptide
SIINFEKL

SEQ ID NO: 6 - DNA, 32 bp, Artificial Sequence
  Primer 5′ Not1
taaatattgcggccgcatggaaccgatcagtg

SEQ ID NO: 7 - DNA, 30 bp, Artificial Sequence
  Primer 3′ Sal1
gattgtcgacttagctggagtgaaaactgg

(19) United States
(12) Patent Application Publication
(10) Pub. No.: US 2018/0371412 A1
(43) Pub. Date: Dec. 27, 2018

(54) THERAPEUTIC T CELLS

(71) Applicant: UCL BUSINESS PLC, London (GB)

(72) Inventors: Ronjon CHAKRAVERTY, London (GB); Emma MORRIS, London (GB)

(21) Appl. No.: 16/062,590

(22) PCT Filed: Dec. 15, 2016
(86) PCT No.: PCT/GB2016/053951

§ 371 Date: Jun. 14, 2018

(30) Foreign Application Priority Data
Dec. 16, 2015 (GB) 1522223.5

(51) Int. Cl. C12N 5/0783; C07K 14/715; A61K 35/17; A61K 38/17; A61P 35/00

(52) U.S. Cl. CPC C12N 5/0636; C07K 14/7158; C12N 2510/00; A61K 38/1793; A61P 35/00; A61K 35/17

(57) ABSTRACT

Use of C-X-C chemokine receptor type 4 (CXCR4) for increasing the capacity for self-renewal and/or persistence in a T cell; increasing the capacity for engraftment in a T cell; and/or increasing the memory function of a T cell.

### FIELD OF THE INVENTION

The present invention relates to methods and compositions for use in immunotherapy. More specifically, the present invention relates to the genetic engineering of T cells to improve their utility in immunotherapy, for example by increasing their capacity for self-renewal and persistence.

### BACKGROUND TO THE INVENTION

Immunotherapy based on the adoptive transfer of naturally occurring or gene-engineered antigen (Ag)-specific T cells represents a highly effective and potentially curative systemic therapy for a number of diseases, including cancer. Melanoma, leukaemias and viral-associated malignancies are particularly responsive to this type of therapy, and successes in these fields have driven attempts to employ this approach against many cancer types.

Immunotherapy approaches may involve optional genetic manipulation of a population of T cells, followed by ex vivo expansion and re-infusion into patients. However, central to the efficacy of these techniques is the success of engraftment and the persistence of the transferred cells—the survival, growth and reproduction of cells within the host recipient following reinfusion.

The efficacy of immunotherapy, for example in anti-tumour treatment, has been shown to be related to the persistence of the transferred T cells in several early phase trials.

Currently, patients need to undergo conditioning with chemotherapy or radiotherapy prior to adoptive transfer of re-directed T cells to ensure adequate engraftment. This approach is associated with significant toxicity and cost. The toxicity is particularly problematic, because patients under consideration for immunotherapy may already be seriously ill. The current need for the aggressive conditioning treatments may therefore prevent the use of immunotherapy or decrease its chances of success by further weakening the patient.

Accordingly, there is a need for providing T cells with the capacity for self-renewal and persistence, so-called T memory stem cells (T_{MSC}), and developing tools to generate them in vitro.

Previous attempts at achieving this aim have faced various setbacks. For example, inhibitors of the WNT pathway have been used in this context, but have led to problems associated with the prevention of cell division. Furthermore, the use of rapamycin and mTOR inhibitors has resulted in observations of a failure of transplanted cells to expand following transfer to a patient.

### SUMMARY OF THE INVENTION

The present inventors have surprisingly found that genetically engineering T cells to express the homing receptor CXCR4 confers the desirable self-renewal and persistence properties of memory stem cells (T_{MSC}). The genetic engineering may, for example, be accomplished by retroviral gene transfer for the provision of inducible or constitutive overexpression of CXCR4.

Specifically, the inventors have found that the engineered T cells engraft via an IL-15-dependent mechanism with high efficiency in non-conditioned recipients as CD44^{low}CD62L^{high} cells. Upon antigen exposure these cells maintain a CD62L^{high} phenotype and show high expression of the anti-apoptotic protein, Bcl-2. This approach increases engraftment of transferred cells and extends their survival long-term compared to controls, and also increases anti-tumour efficacy.

The use of this methodology during clinical engraftment of T cells will negate the current requirement for expensive and debilitating chemotherapy and/or radiotherapy patient conditioning prior to adoptive transfer.

This strategy developed by the inventors provides a cross-platform solution to the problems of engraftment and persistence of therapeutic T cells. This approach can be applied to many types of T cells, including those genetically engineered to express other advantageous components, such as T cell receptors (TCRs) and chimeric antigen receptors (CARs).

Moreover, the invention may provide a 1-shot strategy for therapeutic T cell transfer, in which only one administration is required to achieve long term therapeutic effect. This reduces the impact on a patient of multiple clinical procedures and may also provide advantages in overcoming tumour-editing during the treatment of cancer.

Accordingly, the invention relates to the use of CXCR4 for inducing stemness in a T cell.

In one aspect, the invention provides the use of CXCR4 for:
(a) increasing the capacity for self-renewal and/or persistence in a T cell;
(b) increasing the capacity for engraftment in a T cell; and/or
(c) increasing the memory function of a T cell.

In one embodiment, the invention provides the use of CXCR4 for increasing the capacity for engraftment in a T cell. In another embodiment, the invention provides the use of CXCR4 for increasing the memory function of a T cell. Preferably, the invention provides the use of CXCR4 for increasing the capacity for self-renewal and/or persistence in a T cell.

In one embodiment, the T cell is genetically engineered to express the CXCR4.

In one embodiment, the T cells of the invention persist in a recipient for at least 1, 2, 3, 4, 5, 6, 12, 24, 36, 48 or 72 months longer than T cells that have not been genetically engineered to express CXCR4. In another embodiment, the T cells of the invention persist in a recipient for at least 6 months longer than T cells that have not been genetically engineered to express CXCR4. In another embodiment, the T cells of the invention persist in a recipient for at least 12 months longer than T cells that have not been genetically engineered to express CXCR4. Preferably, the T cells of the invention persist in a recipient for at least 24 months longer than T cells that have not been genetically engineered to express CXCR4.

In another embodiment, the T cells of the invention persist in a recipient in a form expressing CD62L for at least 1, 2, 3, 4, 5, 6, 12, 24, 36, 48 or 72 months longer than T cells that have not been genetically engineered to express CXCR4. In another embodiment, the T cells of the invention persist in a recipient in a form expressing CD62L for at least 6 months longer than T cells that have not been genetically engineered to express CXCR4. In another embodiment, the T cells of the invention persist in a recipient in a form expressing CD62L for at least 12 months longer than T cells that have not been genetically engineered to express CXCR4. In another embodiment, the T cells of the invention persist in a recipient in a form expressing CD62L for at least 24 months longer than T cells that have not been genetically engineered to express CXCR4.

In one embodiment, the T cell is transduced or transfected with a vector comprising a polynucleotide encoding the CXCR4. The vector may be a viral vector, for example a retroviral, adenoviral or adeno-associated viral vector. Preferably, the vector is a retroviral vector, more preferably a lentiviral vector.

In one embodiment, the CXCR4 expression is permanent (i.e. continues throughout the life of a cell). In another embodiment, the CXCR4 expression is temporary, for example detectable CXCR4 expression occurs for less than 4, 3 or 2 weeks, or 7, 6, 5, 4, 3, 2 or 1 days. Expression of the CXCR4 may be controlled using a constitutive or inducible promoter (e.g. the Tet-ON system).

In one embodiment, the CXCR4 is human CXCR4.

In one embodiment, the CXCR4 of the invention:
(a) is encoded by a polynucleotide comprising a nucleotide sequence that has at least 70% identity to SEQ ID NO: 1 or 3, preferably wherein the protein encoded by the nucleotide sequence substantially retains the natural function of the protein represented by SEQ ID NO: 2 or 4, respectively; and/or
(b) comprises a protein that has at least 70% identity to SEQ ID NO: 2 or 4, preferably wherein the amino acid sequence substantially retains the natural function of the protein represented by SEQ ID NO: 2 or 4, respectively.

In one embodiment, the T cell has been further genetically engineered to express a T cell receptor (TCR) and/or chimeric antigen receptor (CAR). The TCR may be an engineered TCR, for example a TCR that has been engineered to increase its recognition of and/or binding affinity towards a target peptide (e.g. a peptide derived from a cancer cell or a virally-infected cell).

In another aspect, the invention provides the use of CXCR4 for preparing a T memory stem cell (T_{MSC}).

In another aspect, the invention provides a method of:
(a) increasing the capacity for self-renewal and/or persistence in a T cell;
(b) increasing the capacity for engraftment in a T cell; and/or
(c) increasing the memory function of a T cell,
   wherein the method comprises the step of genetically engineering the T cell to express CXCR4.

In one embodiment, the invention provides a method of increasing the capacity for engraftment in a T cell, wherein the method comprises the step of genetically engineering the T cell to express CXCR4. In another embodiment, the invention provides a method of increasing the memory function of a T cell, wherein the method comprises the step of genetically engineering the T cell to express CXCR4. Preferably, the invention provides a method of increasing the capacity for self-renewal and/or persistence in a T cell, wherein the method comprises the step of genetically engineering the T cell to express CXCR4.

In another aspect, the invention provides a method of inducing stemness in a T cell, wherein the method comprises the step of genetically engineering the T cell to express CXCR4.

In another aspect, the invention provides a method of preparing a T memory stem cell (T_{MSC}) comprising the step of genetically engineering a T cell to express CXCR4.

In the above-mentioned methods of the invention, the persistence of the T cell, process of genetic engineering, CXCR4 and further characteristics of the T cell may be as described herein.

In another aspect, the invention provides a genetically engineered T cell obtainable through the use of the invention or by the method of the invention. Thus, the invention provides a T cell that has been genetically engineering to express CXCR4.

In one embodiment, the genetically engineered T cell has an increased capacity for self-renewal and/or persistence.

In one embodiment, the genetically engineered T cell has an increased capacity for engraftment.

In one embodiment, the genetically engineered T cell has an increased memory function.

In one embodiment, the T cell has been genetically engineered to express CXCR4.

The increased capacity for self-renewal and/or persistence; engraftment; and/or memory function may be in comparison to a natural T cell or T cell that has not been genetically engineered to express CXCR4.

In one embodiment, the T cell has been further genetically engineered to express a T cell receptor (TCR) and/or chimeric antigen receptor (CAR). The TCR and/or CAR may be as described herein.

In another aspect, the invention provides a genetically engineered T cell which possesses induced stemness.

In another aspect, the invention provides a genetically engineered T cell which has been engineered to become a T memory stem cell (T_{MSC}).

The persistence of the T cell, process of genetic engineering, CXCR4 and further characteristics of the T cell may be as described herein.

In another aspect, the invention provides a pharmaceutical composition comprising the genetically engineered T cell of the invention and a pharmaceutically acceptable carrier, diluent or excipient.

In another aspect, the invention provides a genetically engineered T cell according to the invention for use in therapy.

In another aspect, the invention provides a genetically engineered T cell according to the invention for use in the treatment of cancer.

In one embodiment, the cancer is a melanoma, leukaemia or viral-associated malignancy.

In another aspect, the invention provides a genetically engineered T cell according to the invention for use in the treatment of a viral infection. The viral infection may, for example, be a cytomegalovirus (CMV) infection, Epstein-Barr virus (EBV) infection, human immunodeficiency virus (HIV) infection, adenovirus infection or hepatitis B virus (HBV) infection.

In one embodiment, the subject to be treated is not conditioned before administration of the T cell. For example, the subject to be treated does not undergo chemotherapy or radiotherapy conditioning before administration of the T cell.

In one embodiment, the subject to be treated has not undergone conditioning (e.g. chemotherapy or radiotherapy conditioning) in a period of less than 12, 11, 10, 9, 8, 7, 6, 5, 4, 3, 2 or 1 month(s), or 3, 2 or 1 week(s) before administration of the T cell. In a preferred embodiment, the subject to be treated has not undergone conditioning (e.g. chemotherapy or radiotherapy conditioning) in a period of less than 1 month, or 3, 2 or 1 week(s) before administration of the T cell.

In one embodiment, the T cells are administered in a single dose. The present invention provides therapeutic T cells for which may not need to be re-administered in order to successfully treat a disease.

In another aspect, the invention provides a genetically engineered T cell according to the invention for use in engrafting a subject with T cells.

In another aspect, the invention provides the use of a genetically engineered T cell according to the invention for the manufacture of a medicament for use in therapy. The medicament may be for the treatment of cancer or a viral infection.

In another aspect, the invention provides a method of engrafting a subject with T cells, comprising the steps:
(a) providing a T cell which has been genetically engineered to express CXCR4; and
(b) administering the T cell provided by step (a) to the subject,
   preferably wherein the subject is not conditioned before administration of the T cell.

In another aspect, the invention provides a method of treating or preventing cancer, comprising the steps:
(a) providing a T cell which has been genetically engineered to express CXCR4; and
(b) administering the T cell provided by step (a) to a subject in need thereof,
   preferably wherein the subject to be treated is not conditioned before administration of the T cell.

In one embodiment, the cancer is a melanoma, leukaemia or viral-associated malignancy.

In another aspect, the invention provides a method of treating or preventing a viral infection, comprising the steps:
(a) providing a T cell which has been genetically engineered to express CXCR4; and
(b) administering the T cell provided by step (a) to a subject in need thereof,
   preferably wherein the subject to be treated is not conditioned before administration of the T cell.

In one embodiment of the methods of treatment of the invention, the genetically engineered T cell provided by step (a) has been further genetically engineered to express a T cell receptor (TCR) and/or chimeric antigen receptor (CAR).

In another embodiment, the subject to be treated does not undergo chemotherapy or radiotherapy conditioning before administration of the T cell.

In another embodiment, the subject to be treated has not undergone conditioning (e.g. chemotherapy or radiotherapy conditioning) in a period of less than 12, 11, 10, 9, 8, 7, 6, 5, 4, 3, 2 or 1 month(s), or 3, 2 or 1 week(s) before administration of the T cell. In a preferred embodiment, the subject to be treated has not undergone conditioning (e.g. chemotherapy or radiotherapy conditioning) in a period of less than 1 month, or 3, 2 or 1 week(s) before administration of the T cell.

In another embodiment, the T cells are administered in a single dose.

The persistence of the T cell, process of genetic engineering, CXCR4 and further characteristics of the T cell may be as described herein.

### DESCRIPTION OF THE DRAWINGS

FIG. 1—Engraftment of T cells overexpressing CXCR4 in unconditioned, wild type mice.

(a) Flow cytometric plots showing GFP versus CXCR4 expression in mock transduced cells vs. T^{CXCR4} (pMP71-CXCR4-IRES-GFP) vs. T^{CONTROL} (pMP71-IRES-GFP). (b) CD8⁺ T cells from B6 strain mice were transduced with pMP71-CXCR4-IRES-GFP (CD45.1⁺ T^{CXER4}) or with pMP71-IRES-GFP (Thy1.1⁺ T^{CONTROL}) and mixed at a 1:1 ratio before transfer to non-irradiated B6 CD45.2 mice. Mice were sacrificed 7 days later. The ratio of T^{CXCR4} to T^{CONTROL} was calculated following gating of CD8⁺GFP⁺ cells and calculation of the ratio of CD45.1⁺ to Thy1.1⁺ cells in each organ (BM—bone marrow; Sp—spleen; LN—lymph node). Dotted line indicates ratio of 1.0. Data are pooled from 3 independent experiments (n=11). Statistical comparison was performed using a one-tailed t-test against a theoretical mean of 1.

FIG. 2—Vaccination response in T cells overexpressing CXCR4.

OT-I CD8⁺ T cells (specific for the ovalbumin-derived peptide, SIINFEKL; SEQ ID NO: 5) were transduced with pMP71-CXCR4-IRES-GFP (CD45.2⁺ T_{CXCR4}) or with pMP71-IRES-GFP (CD45.1⁺ T^{CONTROL}) and mixed at a 1:1 ratio before transfer to Rag−/− mice. Mice received a 1° vaccination with SIINFEKL in IFA at day 1 followed by a 2° vaccination (SIINFEKL in IFA) at day 29. At day 37, mice were sacrificed and spleens (Sp), lymph node (LN) and bone marrow (BM) harvested. The graph shows ratio of T^{CXCR4} to T^{CONTROL} at timed intervals following adoptive transfer in each organ. Dotted line indicates a ratio of 1.0.

FIG. 3—Phenotype of T cells overexpressing CXCR4 following vaccination.

(a) Experimental plan as set out in FIG. 2. Mice received BrdU in drinking water for 7 days from day 29. At day 37, mice were sacrificed and spleens (Sp), lymph node (LN) and bone marrow (BM) harvested. Representative flow cytometric histograms show Bcl2, CD122 and BrdU staining of CD8⁺GFP⁺ T cells in the spleen using CD45.2 and CD45.1 to identify T^{CXCR4} and T^{CONTROL}, respectively. Figures top right relate to median fluorescence index (T^{CXCR4}; T^{CONTROL}; neg; respectively top to bottom). (b) Representative flow cytometric histogram shows CD62L staining upon T^{CXCR4} vs. T^{CONTROL} Control staining is of naïve T cells in non-vaccinated mice. Figures top right relate to median fluorescence index (T^{CXCR4}; T^{CONTROL}; T^{NAIVE}; respectively top to bottom). Data are representative of 2 independent experiments.

FIG. 4—Anti-tumour effects of T^{CXCR4} versus T^{CONTROL}.

(a) BALB/c mice were lethally irradiated (8Gy) and reconstituted with B6 bone marrow. Following irradiation, mice were inoculated subcutaneously with 5×10⁶ A20-human CD34 (A20-hCD34) leukaemia cells on day 0. On day +2, mice were given 1.0×10⁵ polyclonal B6 CD8⁺ or T^{CXCR4} or T^{CONTROL} or no T cells, and the size of the tumour documented thereafter (n=5 mice each group). (b) Experimental design as in FIG. 4(a), except that 5×10⁵ A20 leukaemia cells were given by intra-tibial injection on day 0, before intravenous injection of 0.5-1.0×10⁵ polyclonal B6 CD8+T^{CXCR4} or T^{CONTROL} or no T cells on day +2. A20-hCD34 cells were counted (using the human CD34 marker) in the BM on day +11 (n=4 each group) and day +18 (n=9 each group) following bone marrow transplant. Data are pooled from 2 independent experiments. Statistical comparisons were performed using the two-tailed unpaired t-test; * p<0.05, ** p<0.01.

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

The uses and methods of the invention provide T cells that have acquired at least some of the beneficial properties of T memory stem cells (T_{MSC}), from a therapeutic perspective, in particular the capacity for self-renewal and/or persistence. Accordingly, the invention provides the use of CXCR4 for, and methods of, inducing stemness in a T cell.

The term “stemness” refers to characteristics of a cell that are typically associated with a stem cell, for example the ability to differentiate into specific cellular lineages and/or the ability to self renew. The T cells of the invention may have been induced to become more like, or to substantially become T memory stem cells. Accordingly, the induction of stemness in a T cell may refer to the provision of a T cell that has the ability to differentiate, for example into a central memory T cell or an effector memory T cell, and the ability to self renew.

The term “self renewal” refers to the ability of a cell to undergo multiple cycles of cell division while maintaining an undifferentiated state.

T cells with induced stemness may retain the CD62L marker, which distinguishes them from other T cells which shed this marker over time. The retention of CD62L may be indicative of the T cells remaining as a naïve phenotype.

The term “persistence” refers to the ability of the transplanted cells to survive long term in a recipient. For example, persistence may refer to the number of cells descended from the transplanted cells that are detected in the final in vivo evaluation that is conducted at the end of a typical experiment, clinical trial or therapeutic protocol. In one embodiment, persistence is assessed at about 1-72 months, 1-48 months, 1-24 months or 1-12 months after transplantation. In an another embodiment, persistence is assessed at about 1, 2, 3, 4, 5, 6, 12, 24, 36, 48, 60 or 72 months after transplantation.

Persistence may correlate with the efficacy of a therapeutic T cell transplant in the treatment of a disease, for example cancer or a viral infection. The greater the persistence of therapeutic T cells, the more likely a therapeutic regime is to be effective, for example the less likely a tumour relapse will occur.

In one embodiment, the T cells of the invention persist in a recipient for at least 1, 2, 3, 4, 5, 6, 12, 24, 36, 48 or 72 months longer than T cells that have not been genetically engineered to express CXCR4. Preferably, said T cells have not further differentiated.

In another embodiment, the T cells of the invention persist in a recipient in a form expressing CD62L for at least 1, 2, 3, 4, 5, 6, 12, 24, 36, 48 or 72 months longer than T cells that have not been genetically engineered to express CXCR4. Preferably, said T cells have not further differentiated.

The T cells of the invention may also possess an increased ability to engraft in a recipient. In particular, the T cells of the invention may possess an increased ability to engraft in a non-conditioned recipient (e.g. a recipient who has not undergone chemotherapy and/or radiotherapy conditioning).

The term “engraftment” refers to the ability of the transplanted cells to populate a recipient and survive in the immediate aftermath of their transplantation. Accordingly, engraftment is assessed in the short term after transplantation. For example, engraftment may refer to the number of cells descended from the transplanted cells that are detected in the first in vivo evaluation of an experiment, clinical trial or therapeutic protocol, e.g. at the earliest time point that transplanted cells or their descendants may be detected in a recipient. In one embodiment, engraftment is assessed at 0-12, 0-24, 0-48 or 0-72 h after transplantation. In another embodiment, engraftment is assessed at about 1, 2, 3, 4, 5, 6, 12, 24, 36, 48, 60 or 72 h after transplantation. In a preferred embodiment, engraftment is assessed at about 12 h after transplantation.

Persistence and engraftment may be assessed using methods for quantifying cells in vivo that are known in the art. For example, transplanted cells may be genetically engineered to express a marker, for example a reporter protein (e.g. GFP or a surface tag) or DNA sequence, which can be detected ex vivo and used to quantify the numbers of the transplanted cells and their descendents. Cells may be analysed directly from peripheral blood or samples may be extracted from relevant tissues (e.g. bone marrow, lymph nodes and/or spleen) and analysed ex vivo (e.g. by flow cytometry or by polymerase chain reaction).

The term “memory function” refers to a diverse array of behaviours acquired by antigen-experienced T cells that survive following an initial primary response; these include but are not limited to increased basal proliferation and survival in the absence of antigen, a lower threshold for activation following a subsequent antigen encounter and rapid responsiveness (in terms of proliferation, cytokine generation and cytotoxicity). A small subset of memory stem cells can differentiate into other memory cells (e.g. T_{CM} or T_{EM}) upon antigen recognition, while retaining the capacity for self-renewal.

### C-X-C Chemokine Receptor Type 4 (CXCR4)

The T cells of the invention have been genetically engineered to express C-X-C chemokine receptor type 4 (CXCR4).

The term “genetically engineered” refers to the manipulation of a precursor cell, for example a natural cell, by the introduction of exogenous genetic material. Accordingly, in the context of the present invention a T cell may be genetically engineered by the introduction of genetic material that encodes and enables the expression of exogenous CXCR4 by the cell.

CXCR4 is a homing receptor that binds to CXCL12 and is involved in regulation of cell trafficking to the bone marrow and lymph nodes.

CXCR4 may also known as fusin or CD184.

In a preferred embodiment of the invention, the CXCR4 is human CXCR4.

In one embodiment, the nucleotide sequence encoding CXCR4 is the sequence deposited under NCBI Accession No. NM_003467.2.

In another embodiment, the nucleotide sequence encoding CXCR4 is:

(SEQ ID NO: 1 - Human CXCR4)
ATGGAGGGGATCAGTATATACACTTCAGATAACTACACCGAGGAAATGGG
CTCAGGGGACTATGACTCCATGAAGGAACCCTGTTTCCGTGAAGAAAATG
CTAATTTCAATAAAATCTTCCTGCCCACCATCTACTCCATCATCTTCTTA
ACTGGCATTGTGGGCAATGGATTGGTCATCCTGGTCATGGGTTACCAGAA
GAAACTGAGAAGCATGACGGACAAGTACAGGCTGCACCTGTCAGTGGCCG
ACCTCCTCTTTGTCATCACGCTTCCCTTCTGGGCAGTTGATGCCGTGGCA
AACTGGTACTTTGGGAACTTCCTATGCAAGGCAGTCCATGTCATCTACAC
AGTCAACCTCTACAGCAGTGTCCTCATCCTGGCCTTCATCAGTCTGGACC
GCTACCTGGCCATCGTCCACGCCACCAACAGTCAGAGGCCAAGGAAGCTG
TTGGCTGAAAAGGTGGTCTATGTTGGCGTCTGGATCCCTGCCCTCCTGCT
GACTATTCCCGACTTCATCTTTGCCAACGTCAGTGAGGCAGATGAGAGAT
ATATCTGTGACCGCTTCTACCCCAATGACTTGTGGGTGGTTGTGTTCCAG
TTTCAGCACATCATGGTTGGCCTTATCCTGCCTGGTATTGTCATCCTGTC
CTGCTATTGCATTATCATCTCCAAGCTGTCACACTCCAAGGGCCACCAGA
AGCGCAAGGCCCTCAAGACCACAGTCATCCTCATCCTGGCTTTCTTCGCC
TGTTGGCTGCCTTACTACATTGGGATCAGCATCGACTCCTTCATCCTCCT
GGAAATCATCAAGCAAGGGTGTGAGTTTGAGAACACTGTGCACAAGTGGA
TTTCCATCACCGAGGCCCTAGCTTTCTTCCACTGTTGTCTGAACCCCATC
CTCTATGCTTTCCTTGGAGCCAAATTTAAAACCTCTGCCCAGCACGCACT
CACCTCTGTGAGCAGAGGGTCCAGCCTCAAGATCCTCTCCAAAGGAAAGC
GAGGTGGACATTCATCTGTTTCCACTGAGTCTGAGTCTTCAAGTTTTCAC
TCCAGCTAA

In one embodiment, the amino acid sequence of CXCR4 is the sequence deposited under NCBI Accession No. NP_003458.1.

In another embodiment, the amino acid sequence of CXCR4 is:

(SEQ ID NO: 2 - Human CXCR4)
MEGISIYTSDNYTEEMGSGDYDSMKEPCFREENANFNKIFLPTIYSIIFL
TGIVGNGLVILVMGYQKKLRSMTDKYRLHLSVADLLFVITLPFWAVDAVA
NWYFGNFLCKAVHVTYTVNLYSSVLILAFISLDRYLAIVHATNSQRPRKL
LAEKVVYVGVWIPALLLTIPDFIFANVSEADDRYICDRFYPNDLWVVVFQ
FQHIMVGLILPGIVILSCYCIIISKLSHSKGHQKRKALKTTVILILAFFA
CWLPYYIGISIDSFILLEIIKQGCEFENTVHKWISTTEALAFFHCCLNPI
LYAFLGAKFKTSAQHALTSVSRGSSLKILSKGKRGGHSSVSTESESSSFH
SS

In another embodiment, the CXCR4 is murine CXCR4.

In one embodiment, the nucleotide sequence encoding CXCR4 is the sequence deposited under NCBI Accession No. NM_009911.3.

In another embodiment, the nucleotide sequence encoding CXCR4 is:

(SEQ ID NO: 3 - Murine CXCR4)
ATGGAACCGATCAGTGTGAGTATATACACTTCTGATAACTACTCTGAAGA
AGTGGGTTCTGGAGACTATGACTCCAACAAGGAACCCTGCTTCCGGGATG
AAAACGTCCATTTCAATAGGATCTTCCTGCCCACCATCTACTTCATCATC
TTCTTGACTGGCATAGTCGGCAATGGATTGGTGATCCTGGTCATGGGTTA
CCAGAAGAAGCTAAGGAGCATGACGGACAAGTACCGGCTGCACCTGTCAG
TGGCTGACCTCCTCTTTGTCATCACACTCCCCTTCTGGGCAGTTGATGCC
ATGGCTGACTGGTACTTTGGGAAATTTTTGTGTAAGGCTGTCCATATCAT
CTACACTGTCAACCTCTACACCAGCGTTCTCATCCTGCCCTTCATCAGCC
TGGACCCGTACCTCGCTATTGTCCACGCCACCAACAGTCAGAGGCCAAGG
AAACTGCTGGCTGAAAAGGCAGTCTATGTGGGCGTCTGGATCCCAGCCCT
CCTCCTGACTATACCTGACTTCATCTTTGCCGACGTCAGCCAGGGGGACA
TCAGTCAGGGGGATGACAGGTACATCTGTGACCGCCTTTACCCCGATAGC
CTGTGGATGGTGGTGTTTCAATTCCAGCATATAATGGTGGCTCTCCTCCT
GCCCGGCATCGTCATCCTCTCCTGTTACTGCATCATCATCTCTAACCTGT
CACACTCCAAGGGCCACCAGAAGCGCAAGGCCCTCAAGACGACAGTCATC
CTCATCCTAGCTTTCTTTGCCTGCTGGCTGCCATATTATGTGGGGATCAG
CATCGACTCCTTCATCCTTTTGGGGGTCATCAAGCAAGGATGTGACTTCC
AGAGCATCGTGCACAACTGCATCTCCATCACAGAGGCCCTCGCCTTCTTC
CACTGTTCCCTGAACCCCATCCTCTATGCCTTCCTCGGGGCCAAGTTCAA
AAGCTCTGCCCAGCATGCACTCAACTCCATGAGCAGAGGCTCCAGCCTCA
AGATCCTTTCCAAAGGAAAGCGGGGTGGACACTCTTCCGTCTCCACGGAG
TCAGAATCCTCCAGTTTTCACTCCAGCTAA

In one embodiment, the amino acid sequence of CXCR4 is the sequence deposited under NCBI Accession No. NP_034041.2.

In another embodiment, the amino acid sequence of CXCR4 is:

(SEQ ID NO: 4 - Murine CXCR4)
MEPISVSIYTSDNYSEEVGSGDYDSNKEPCFRDENVHFNRIFLPTIYFII
FLTGIVGNGLVILVMGYQKKLRSMTDKYRLHLSVADLLFVITLPFWAVDA
MADWYFGKFLCKAVHIIYTVNLYSSVLILAFISLDRYLAIVHATNSQRPR
KLLAEKAVYVGVWIPALLLTIPDFIFADVSQGDISQGDDRYICDRLYPDS
LWMVVFQFQHIMVGLVLPGIVILSCYCIIISKLSHSKGHQKRKALKTTVI
LILAFFACWLPYYVGISIDSFILLGVIKQGCDFESIVHKWISITEALAFF
HCCLNPILYAFLGAKFKSSAQHALNSMSRGSSLKILSKGKRGGHSSVSTE
SESSSFHSS

The nucleotide sequence encoding CXCR4 of the invention may, for example, comprise a nucleotide sequence that has at least 70%, 80%, 90%, 95%, 96%, 97%, 98% 99% or 100% identity to SEQ ID NO: 1 or 3, wherein the protein encoded by the nucleotide sequence substantially retains the natural function of the protein represented by SEQ ID NO: 2 or 4, respectively.

The nucleotide sequence encoding CXCR4 of the invention may, for example, encode an amino acid sequence that has at least 70%, 80%, 90%, 95%, 96%, 97%, 98% 99% or 100% identity to SEQ ID NO: 2 or 4, wherein the amino acid sequence substantially retains the natural function of the protein represented by SEQ ID NO: 2 or 4, respectively.

The CXCR4 amino acid sequence of the invention may, for example, comprise or consist of a sequence that has at least 70%, 80%, 90%, 95%, 96%, 97%, 98% 99% or 100% identity to SEQ ID NO: 2 or 4, wherein the amino acid sequence substantially retains the natural function of the protein represented by SEQ ID NO: 2 or 4, respectively.

Preferably, the CXCR4 amino acid sequence of the invention provides a similar or higher:
(a) increase in the capacity for self-renewal and/or persistence in a T cell; and/or
(b) induction of stemness in a T cell,
   when expressed in the T cell, as the protein of SEQ ID NO: 2 or 4.

### T Cell

T cells (or T lymphocytes) are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Cytotoxic T cells (T_{C} cells or CTLs) destroy virally infected cells and tumour cells, and are also implicated in transplant rejection. T_{C} cells express CD8 at their surface. These cells recognise their targets by binding to antigen associated with MHC class I, which is present on the surface of all nucleated cells.

Helper T helper cells (T_{H} cells) assist other white blood cells in immunologic processes, including maturation of B cells into plasma cells and memory B cells, and activation of cytotoxic T cells and macrophages. T_{H} cells express CD4 on their surface. T_{H} cells become activated when they are presented with peptide antigens by MHC class II molecules on the surface of antigen presenting cells (APCs). These cells can differentiate into one of several subtypes, including T_{H}1, T_{H}2, T_{H}3, T_{H}17, Th9 or T_{FH}, which secrete different cytokines to facilitate different types of immune responses.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells may be either CD4⁺ or CD8⁺ and typically express the cell surface protein CD45RO.

Memory T cells comprise three subtypes: central memory T cells (T_{CM} cells); effector memory T cells (T_{EM} cells); and T memory stem cells (T_{MSC}).

T memory stem cells (T_{MSC}) are characterised by the expression of naïve-like markers (e.g. CD45RA⁺, CCR7⁺, CD27⁺, CD28⁺, CD62L⁺, CD127⁺), together with other markers (e.g. CD122, CXCR3 and CD95) observed in antigen-experienced cells.

Regulatory T cells (T_{reg} cells), formerly known as suppressor T cells, are crucial for the maintenance of immunological tolerance. Their major role is to shut down T cell-mediated immunity towards the end of an immune reaction and to suppress auto-reactive T cells that escaped the process of negative selection in the thymus.

Two major classes of CD4⁺ T_{reg} cells have been described—naturally occurring T_{reg} cells and adaptive T_{reg} cells.

Naturally occurring T_{reg} cells (also known as CD4⁺CD25⁺FOXP3⁺ T_{reg} cells) arise in the thymus and have been linked to interactions between developing T cells with both myeloid (CD11c⁺) and plasmacytoid (CD123⁺) dendritic cells that have been activated with TSLP. Naturally occurring T_{reg} cells can be distinguished from other T cells by the presence of an intracellular molecule called FOXP3. Mutations of the FOXP3 gene can prevent regulatory T cell development, causing the fatal autoimmune disease IPEX.

Adaptive T_{reg} cells (also known as Tr1 cells or Th3 cells) may originate during a normal immune response.

The T cell of the invention may be any of the T cell types mentioned above. Preferably, cytotoxic T cells (T_{C} cells).

The CXCR4-expressing T cells of the invention may be generated by introducing DNA or RNA encoding the CXCR4 by one of many means known in the art, for example transduction with a viral vector or transfection with DNA or RNA.

The invention also provides a population of cells comprising the CXCR4-expressing T cells of the invention. The population of cells may, for example, be prepared by transducing or transfecting a blood-sample ex vivo with a vector comprising a polynucleotide encoding CXCR4.

CXCR4-expressing T cells of the invention may be created ex vivo from a patient's own peripheral blood (1^{st} party), or in the setting of a haematopoietic stem cell transplant from donor peripheral blood (2^{nd} party), or peripheral blood from an unconnected donor (3^{rd} party).

Alternatively, CXCR4-expressing T cells may be derived from ex vivo differentiation of inducible progenitor cells or embryonic progenitor cells.

Alternatively, an immortalised cell line such as a T cell line which retains its lytic function and could act as a therapeutic may be used.

The invention may relate to ex vivo or in vitro, preferably ex vivo, genetic engineering of a T cell. A T cell of the invention may be an ex vivo T cell from a subject. The T cell may be from a peripheral blood mononuclear cell (PBMC) sample. T cells may be activated and/or expanded prior to being transduced with a CXCR4-encoding nucleic acid, for example by treatment with an anti-CD3 monoclonal antibody.

A CXCR4-expression T cell of the invention may be prepared by:
(a) isolating a T cell-containing sample from a subject or other source listed above; and
(b) transducing or transfecting the T cells with one or more polynucleotide(s) encoding the CXCR4.

The T cells may then by purified, for example by selection on the basis of expression of the CXCR4.

The T cells of the invention may be, for example, human or murine T cells. Preferably the T cells are human T cells.

Although the description herein may refer to a T cell, the invention also relates to populations of the T cells of the invention.

### T Cell Receptor (TCR)

The T cells of the invention may also comprise one or more exogenous T cell receptors (TCRs), for example the T cells of the invention may have been genetically modified to express one or more TCRs. Preferably, the TCRs are engineered TCRs.

During antigen processing, antigens are degraded inside cells, and then carried to and displayed on the cell surface by major histocompatability complex (MHC) molecules. Two different classes of MHC molecules, MHC I and MHC II, deliver peptides from different cellular compartments to the cell surface. T cells are able to recognise this peptide:MHC complex at the surface of the antigen presenting cell via their T cell receptors (TCRs).

The TCR is expressed on the surface of T cells and is a heterodimeric protein consisting of an α and β chain in 95% of T cells, or a γ and δ chains in 5% of T cells.

Engagement of the TCR with antigen and MHC results in activation of its T lymphocyte through a series of biochemical events mediated by associated enzymes, co-receptors, and specialised accessory molecules.

Each chain of the TCR is a member of the immunoglobulin superfamily and possesses one N-terminal immunoglobulin (Ig)-variable (V) domain, one Ig-constant (C) domain, a trans-membrane/cell membrane-spanning region and a short cytoplasmic tail at the C-terminal end.

The variable domains of both the TCR α-chain and β-chain have three hypervariable or complementarity determining regions (CDRs). CDR3 is the main CDR responsible for recognising processed antigen, although CDR1 of the alpha chain has also been shown to interact with the N-terminal part of the antigenic peptide, whereas CDR1 of the beta chain interacts with the C-terminal part of the peptide. CDR2 is thought to recognise the MHC molecule. Framework regions (FRs) are positioned between the CDRs. These regions provide the structure of the TCR variable region.

TCRs may associate with other molecules, for example CD3 which possesses three distinct chains (γ, δ and ε) in mammals, and the ζ-chain. These accessory molecules have negatively charged transmembrane regions and are vital to propagating the signal from the TCR into the cell. The CD3- and ζ-chains, together with the TCR, form what is known as the T cell receptor complex.

The TCRs of the invention may be engineered TCRs, for example TCRs that have been artificially mutated to confer improved recognition and binding affinity towards target peptides (e.g. cancer cell- or virus-derived peptides). Such engineered TCRs may further improve the recognition and destruction of cancer cells or virus-infected cells.

TCR-encoding nucleic acids may be transferred to T cells using any suitable means known in the art, for example using retroviral vectors. Lentiviral vectors may be employed. In this way, a large number of specific TCR-expressing T cells can be generated for adoptive cell transfer.

Example TCRs of the invention include TCRs specific for target antigens including tumour-associated antigens, tissue-specific differentiation antigens, cancer testis antigens, tumour-specific antigens, mutated tumour antigens and viral antigens.

### Chimeric Antigen Receptor (CAR)

The T cells of the invention may also comprise one or more chimeric antigen receptors (CARs), for example the T cells of the invention may have been genetically modified to express one or more CARs.

CARs are chimeric type I trans-membrane proteins which connect an extracellular antigen-recognising domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it to adopt a suitable orientation. A common spacer domain used is the Fc of IgG1. More compact spacers can suffice, for example the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T cell killing of cognate target cells but failed to fully activate the T cell to proliferate and survive. To overcome this limitation, compound endodomains have been constructed: fusion of the intracellular part of a T cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal, namely immunological signal 2, which triggers T cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T cell it is expressed in. Thus the CAR directs the specificity and cytotoxicity of the T cell towards, for example, tumour cells expressing the targeted antigen.

CAR-encoding nucleic acids may be transferred to T cells using any suitable means known in the art, for example using retroviral vectors. Lentiviral vectors may be employed. In this way, a large number of specific CAR-expressing T cells, for example cancer-specific T cells, can be generated for adoptive cell transfer.

Example CARs of the invention include CARs specific for target antigens including tumour-associated antigens, tissue-specific differentiation antigens, cancer testis antigens, tumour-specific antigens, mutated tumour antigens and viral antigens.

### Pharmaceutical Composition

In one aspect, the invention provides a pharmaceutical composition comprising a plurality of T cells of the invention.

The pharmaceutical composition may additionally comprise a pharmaceutically acceptable carrier, diluent or excipient. The pharmaceutical composition may optionally comprise one or more further pharmaceutically active compounds, e.g. polypeptides. Such a formulation may, for example, be in a form suitable for intravenous infusion.

### Method of Treatment

The T cells of the invention may be capable of killing target cells, for example cancer cells.

The T cells of the invention may be used for the treatment of an infection, for example a viral infection.

The T cells of the invention may be used for the control of pathogenic immune responses, for example in autoimmune diseases, allergies and in graft-versus-host rejection.

The T cells of the invention may be used for the treatment of a cancerous diseases, for example bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The T cells of the invention are particularly suited for the treatment of solid tumours where the availability of good selective single targets is limited.

The T cells of the invention may be used to treat: cancers of the oral cavity and pharynx which include cancer of the tongue, mouth and pharynx; cancers of the digestive system which include oesophageal, gastric and colorectal cancers; cancers of the liver and biliary tree which include hepatocellular carcinomas and cholangiocarcinomas; cancers of the respiratory system which include bronchogenic cancers and cancers of the larynx; cancers of bone and joints which include osteosarcoma; cancers of the skin which include melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers which include gliomas, glioblastoma multiforme and medullobastomas; cancers of the endocrine system which include thyroid cancer, adrenal carcinoma and cancers associated with multiple endocrine neoplasm syndromes; lymphomas which include Hodgkin's lymphoma and non-Hodgkin lymphoma; Multiple Myeloma and plasmacytomas; leukaemias, both acute and chronic, myeloid or lymphoid; and cancers of other and unspecified sites including neuroblastoma.

Treatment with the T cells of the invention may help prevent the escape or release of tumour cells which often occurs with standard approaches.

The T cells of the invention may be used to treat chronic infections, including cytomegalovirus (CMV) infections, Epstein-Barr virus (EBV) infections, human immunodeficiency virus (HIV) infections, hepatitis B virus (HBV) infections or hepatitis C virus (HCV) infections.

It is to be appreciated that all references herein to treatment include curative, palliative and prophylactic treatment; although in the context of the invention references to preventing are more commonly associated with prophylactic treatment. Treatment may also include arresting progression in the severity of a disease.

The treatment of mammals, particularly humans, is preferred. However, both human and veterinary treatments are within the scope of the invention.

### Conditioning

Typically, a patient must undergo conditioning before the transfer of therapeutic T cells. Such conditioning is required to prepare the patient's immune system to accept the transferred cells and to reduce the risk of the patient's immune system rejecting and destroying the cells.

Conditioning may take the form of chemotherapy and/or radiotherapy treatment.

The present invention overcomes or reduces the need for patient conditioning before the transfer of the therapeutic T cells.

### Vectors

The genetically engineered T cells of the invention may be prepared using vectors to introduce CXCR4 to precursor T cells. The introduction of further proteins (e.g. TCRs and/or CARs) to prepare T cells of the invention may also be achieved using vectors.

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid and/or facilitating the expression of the protein encoded by a segment of nucleic acid.

Vectors may be non-viral or viral. Examples of vectors used in recombinant nucleic acid techniques include, but are not limited to, plasmids, chromosomes, artificial chromosomes and viruses. Vectors may also be, for example, naked nucleic acids (e.g. DNA). In its simplest form, the vector may itself be a nucleotide of interest.

The vectors used in the invention may be, for example, plasmid or viral vectors, and may include a promoter for the expression of a polynucleotide and optionally a regulator of the promoter.

Vectors comprising polynucleotides used in the invention may be introduced into cells using a variety of techniques known in the art, such as transfection, transduction and transformation.

Transfection may refer to a general process of incorporating a nucleic acid into a cell and includes a process using a non-viral vector to deliver a polynucleotide to a cell. Transduction may refer to a process of incorporating a nucleic acid into a cell using a viral vector.

Example techniques for introducing a vector into a cell include infection with recombinant viral vectors (e.g. retroviral, lentiviral, adenoviral, adeno-associated viral, baculoviral and herpes simplex viral vectors); direct injection of nucleic acids and biolistic transfection/transformation; heat shock; electroporation; lipid-mediated transfection; compacted DNA-mediated transfection; use of liposomes, immunoliposomes, lipofectin, cationic facial amphiphiles (CFAs; Nature Biotechnology (1996) 14: 556) and cationic agent-mediated transfection; and combinations thereof.

### Viral Vectors

In one embodiment, a viral vector is used in the invention to introduce a nucleotide of interest (e.g. a polynucleotide that encodes CXCR4, a TCR and/or a CAR) into a cell.

In one embodiment, the viral vector is a retroviral, lentiviral, adenoviral or adeno-associated viral vector. In a preferred embodiment, the viral vector is a retroviral vector, particularly preferably a lentiviral vector.

A specific “viral vector” is a vector which comprises at least one component part derivable from that specific virus. Preferably, that component part is involved in the biological mechanisms by which the vector infects cells, expresses genes or is replicated. Thus, for example, a “lentiviral vector” is a vector that comprises at least one component part derivable from a lentivirus.

Preferably, the viral vector is replication defective. This may be achieved, for example, by removing at least part of one or more protein-coding regions essential for replication from the virus.

### Retroviral and Lentiviral Vectors

In one embodiment, the viral vector is a retroviral vector.

The retroviral vector used in the invention may be derived from or may be derivable from any suitable retrovirus. A large number of different retroviruses have been identified. Examples include: murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo MLV), FBR murine osteosarcoma virus (FBR MSV), Moloney murine sarcoma virus (Mo-MSV), Abelson murine leukaemia virus (A-MLV), Avian myelocytomatosis virus-29 (MC29) and Avian erythroblastosis virus (AEV). A detailed list of retroviruses may be found in Coffin et al. (1997) Retroviruses, Cold Spring Harbor Laboratory Press, Eds: Coffin, J. M., Hughes, S. M., Varmus, H. E., pp. 758-763.

Retroviruses may be broadly divided into two categories, namely “simple” and “complex”. Retroviruses may even be further divided into seven groups. Five of these groups represent retroviruses with oncogenic potential. The remaining two groups are the lentiviruses and the spumaviruses. A review of these retroviruses is presented in Coffin et al. (1997) Retroviruses, Cold Spring Harbor Laboratory Press, Eds: Coffin, J. M., Hughes, S. M., Varmus, H. E., pp. 758-763.

In another embodiment, the viral vector is a retroviral vector.

A detailed list of lentiviruses may also be found in Coffin et al. (1997) Retroviruses, Cold Spring Harbor Laboratory Press, Eds: Coffin, J. M., Hughes, S. M., Varmus, H. E., pp. 758-763. In brief, lentiviruses can be divided into primate and non-primate groups. Examples of primate lentiviruses include but are not limited to: the human immunodeficiency virus (HIV; the causative agent of human acquired immune deficiency syndrome, AIDS), and the simian immunodeficiency virus (SIV). Non-primate lentiviruses includes the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis et al. (1992) EMBO J. 11: 3053-3058 and Lewis and Emerman (1994) J. Virol. 68: 510-516). In contrast, other retroviruses, such as MLV, are unable to infect non-dividing or slowly dividing cells such as those that make up, for example, muscle, brain, lung and liver tissue.

### Adenoviral Vectors

In one embodiment, the vector is an adenoviral vector.

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Adeno-Associated Virus Vectors

In one embodiment, the vector is an adeno-associated viral (AAV) vector.

AAV has a high frequency of integration and can infect non-dividing cells. This makes it useful for delivery of genes into mammalian cells in tissue culture. AAV has a broad host range for infectivity.

Recombinant AAV vectors have been used successfully for in vitro and in viva transduction of marker genes and genes involved in human diseases.

### Variants, Derivatives, Analogues, Homologues and Fragments

In addition to the specific proteins and nucleotides mentioned herein, the invention also encompasses the use of variants, derivatives, analogues, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring polypeptide or polynucleotide.

The term “derivative” as used herein, in relation to proteins or polypeptides of the invention includes any substitution, variation, modification, replacement, deletion and/or addition of one (or more) amino acid residues from or to the sequence, providing that the resultant protein or polypeptide substantially retains at least one of its endogenous functions.

The term “analogue” as used herein, in relation to polypeptides or polynucleotides of the invention includes any mimetic, i.e. a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column, and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein, means an entity having a certain homology with the wild type amino acid sequence and the wild type nucleotide sequence. The term “homology” may be equated with “identity”.

A homologous sequence may include an amino acid sequence which may be at least 50%, 55%, 60%, 65%, 70%, 75%, 80%, 85% or 90% identical, preferably at least 95% or 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 96% or 97% or 98% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (Ausubel et al. (1999) ibid, Ch. 18), FASTA (Atschul et al. (1990) J. Mol. Biol. 403-410) and the GENEWORKS suite of comparison tools. Both BLAST and FASTA are available for offline and online searching (Ausubel et al. (1999) ibid, pp. 7-58 to 7-60). However, for some applications it is preferred to use the GCG Bestfit program. Another tool, called BLAST 2 Sequences is also available for comparing protein and nucleotide sequences (FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” of full length CXCR4 are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention, e.g. polynucleotides encoding CXCR4, TCR and/or CAR, may be codon-optimised.

Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### EXAMPLES

### Example 1

### Materials and Methods

Cxcr4 Cloning from Murine BM

Messenger RNA was extracted from murine bone marrow using the Qiagen RNAeasy kit as per manufacturer's instructions. RT-PCR using Invitrogen DNA polymerase and buffers was performed on the isolated mRNA to produce cDNA. The mRNA sequence for murine Cxcr4 was obtained from the online NCBI nucleotide reference library (NCBI Accession No. NM_009911) and primers designed to flank the Cxcr4 coding sequence. The 5′ primer was commenced with the sequence of the Nod restriction endonuclease and 3′ primer with the Sal1 sequence, generating a PCR product with these restriction sites flanking the subsequently amplified DNA. These primers were then used to amplify the CXCR4 DNA:

(SEQ ID NO: 6)
5′ Not1 primer: TAAATATTGCGGCCGCATGGAACCGATCAGTG
(SEQ ID NO: 7)
3′ Sal1 primer: GATTGTCGACTTAGCTGGAGTGAAAACTGG

### CXCR4-GFP Retroviral Vector Production

The murine Cxcr4 gene was cloned into the pMP71 retroviral backbone. After Not1/Sal1 digestion of the Cxcr4 product and the pMP71 vector, the murine Cxcr4 insert was then ligated into the linearised pMP71 backbone using a 10 μl reaction containing 1 μl 10× T4 DNA ligase buffer (New England BioLabs), 0.5 μl (200 U) T4 DNA ligase (New England BioLabs), Cxcr4 insert and linearised pMP71 at a molar ratio of 3:1. The reactions were incubated at 14° C. overnight. This resulted in a construct encoding for CXCR4 and GFP separated by an IRES sequence, denoted as pMP71 CXCR4-IRES-GFP.

### Transfection and Retroviral Particle Production

The Phoenix Eco packaging cell line was used to generate high concentrations of retroviral particles following transient transfection. 1.5×10⁶ cells in 8 ml of packaging cell media were plated out on 10 cm tissue-culture treated plates. 24 h later the media was replaced with 5.5 ml of new IMDM media and after at least 30 min the transfection mixture was pipetted evenly onto the plate. The transfection mixture was produced by adding 10 μl of Fugene-HD transfection reagent (Roche-04709705001) to 300 μl of serum-free Opti-MEM medium followed by 2.6 μg plasmid DNA and 1.5 μg pCl Eco DNA. After another 24 h the media was replaced by 5.5 ml of T cell media. 24 h later, the supernatant was harvested and spun down to remove cellular debris. Transfection efficiency was checked by FACS analysis of the packaging cells to determine the percentage of cells expressing GFP (when the vector contained a GFP reporter). Retroviral production process was identical for the CXCR4 and control to produce CXCR4-GFP or control vector-containing supernatant.

### Retroviral Transduction of T Cells

T cells were resuspended at a concentration of 1×10⁶ per ml in T cell media with 2 μg/ml of concanavalin A (conA) (Sigma-Aldrich) and 1 ng/ml of human IL-7 (R and D Systems). T cells were incubated for 24 h to allow activation prior to transduction. Three hours prior to the transduction, 6 well non-tissue culture-treated plates were coated with RetroNectin (Takara-Bio—Otsu, Japan), and then blocked with 2% bovine serum albumin in PBS for 30 minutes before washing twice in PBS. Up to 6×10⁶ T cells were re-suspended in 1.5 ml of the appropriate transfection supernatant, containing retrovirus as harvested from the packaging cells. This plate was then spun at 1000 g for 90 min with no brake. The following day 4 ml of fresh T cell media was added with IL-2 (Chiron) to achieve a final IL-2 concentration of 100 U/ml IL-2.

### In Vivo T Cell Trafficking

Donor CD8+ T cells were used from B6 mice bearing either a Thy1.1 or CD45.1 congenic marker. These transduced populations were injected intravenously via the tail vein of B6 CD45.2 Thy1.2 mice on day 0.1×10⁶ transduced cells were administered to each mouse, resuspended in sterile PBS. The mice were sacrificed by a schedule 1 technique on day 7 post transfer and organs harvested. Spleen, bone marrow (1× tibia/femur) and lymph nodes (LN-inguinal ×2, brachial ×2 and axilliary ×2) were harvested. Single cell suspensions in FACS buffer were prepared and cell numbers counted ready for FACS staining. BM and spleen samples were resuspended in ACK lysis buffer (Lonza) 1 ml for two minutes and then quenched with 9 ml of FACS buffer to remove red cells, spun down and re-suspended for FACS staining. To interpret the output data the FACS data was analysed using a lymphocyte gated followed by a CD8⁺/adoptive congenic marker gate e.g. CD45.1.

### Results

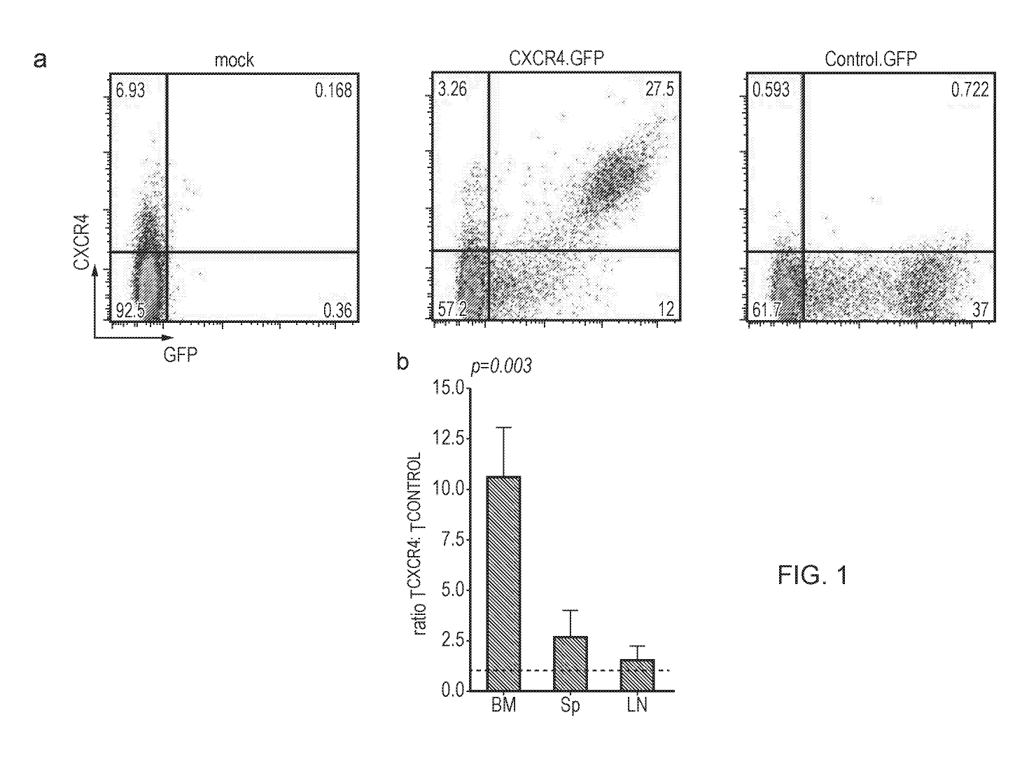

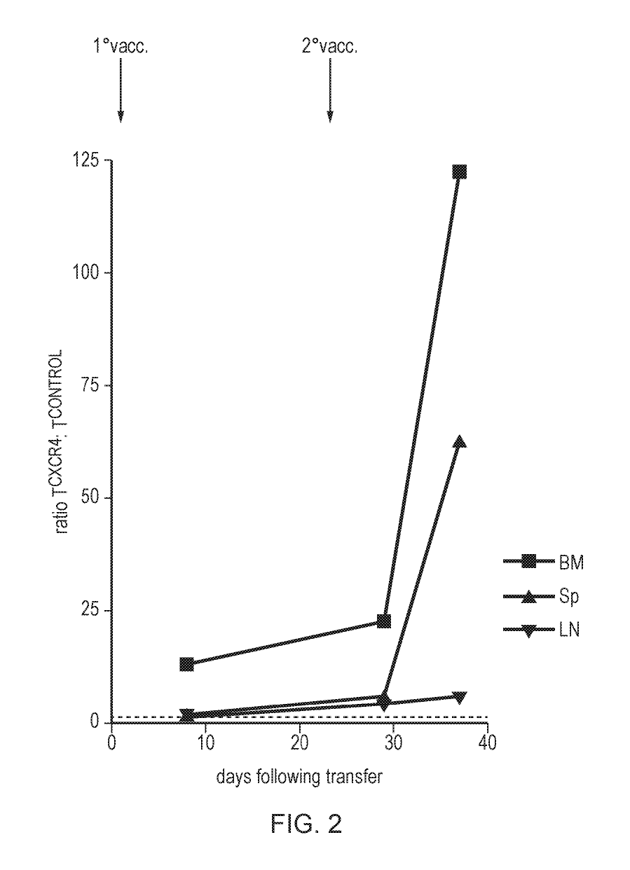

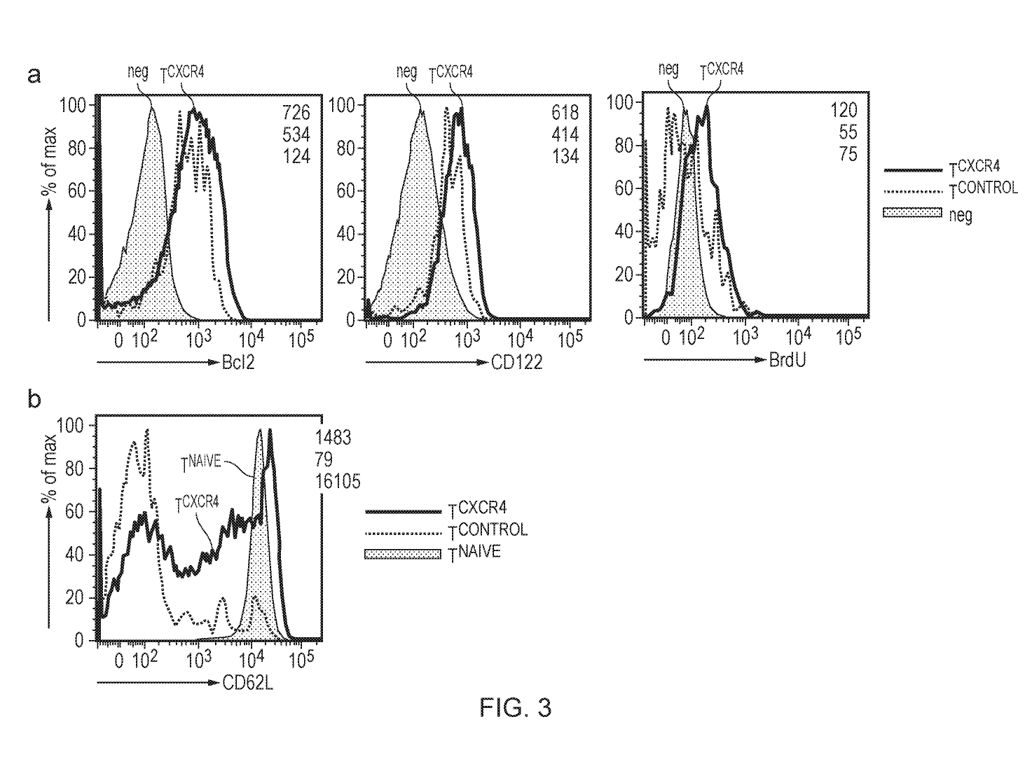

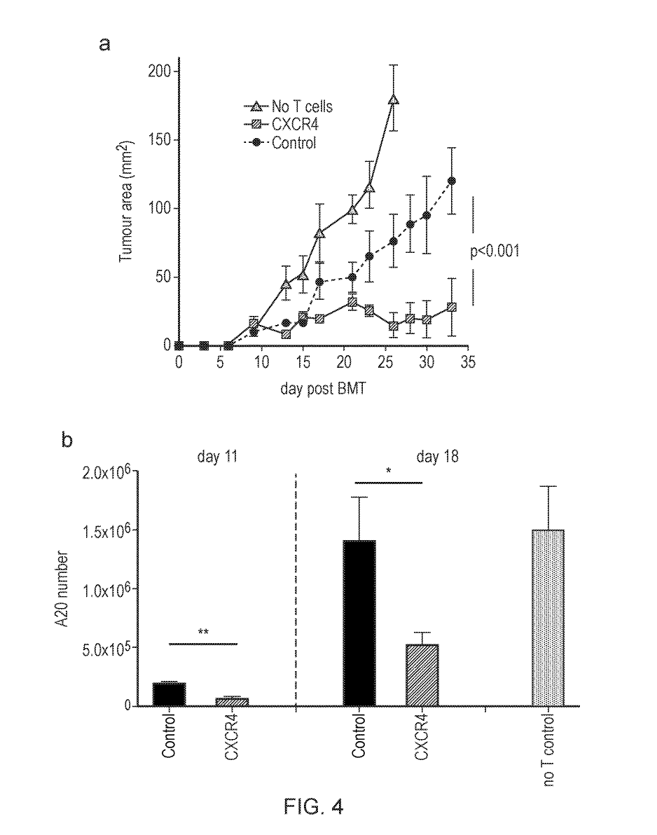

These data confirm increased expression of CXCR4 protein in CD8⁺ T cells transduced with pMP71-CXCR4-IRES-GFP (FIG. 1). Upon transfer to non-conditioned mice, CD8⁺ T cells transduced with pMP71-CXCR4-IRES-GFP demonstrate ˜10-fold mean increase in numbers in the bone marrow compared to control-vector transduced T cells.

### Example 2

### Materials and Methods

OT-1 T cells (either CD45.1+ or Thy1.1+) were transduced with CXCR4-IRES-GFP or control IRES-GFP vectors, mixed at a 1:1 ratio before injection into B6 CD45.2+ Rag−/− mice (1×10⁶ cells for each population). Mice were vaccinated at the base of the tail with 200 μM SIINFEKL (relevant) or irrelevant peptide in incomplete Freund's adjuvant (IFA) on day 1 and day 29. At the time points indicated mice were sacrificed, and BM, spleen and LN harvested, and relative numbers of CXCR4- or control vector-transduced cells were evaluated by FACs.

### Results

These data confirm that OT-1 CD8⁺ T cells transduced with pMP71-CXCR4-IRES-GFP outcompete cells transduced with the vector control in a recall immune response to antigen, particularly in the spleen and bone marrow (FIG. 2). Thus, T^{CXCR4} display better memory properties than control cells.

### Example 3

### Materials and Methods

The experimental plans are as per Example 2. For cell surface staining, cells were plated out at up to 1×10⁶ per well in a 96 well round bottom plate. The cells were re-suspended in 50 μl of FACS buffer (2% FCS in PBS) together with the appropriate concentrations of the indicated fluorochrome-conjugated antibody. The plate was then incubated in the dark at 4° C. for 20 min. The wells were then made up to 200 μl with FACS buffer and washed once more. Stained cells were then resuspended in 200 μl FAGS buffer ready for FACS analysis. For intra-cellular staining, cells were initially stained with surface antibodies as above. Cells were then washed in MACS buffer and in 200 μl of 1% formaldehyde fixation/permeabilisation solution (Cytofix/Cytoperm—BD), incubated at 4° C. for 15 minutes, washed twice in 0.5% saponin (Perm/Wash buffer—BD), re-suspended in 50 μl of Perm/Wash buffer supplemented with fluorochrome-labelled anti-bcl2 antibody or isotype control, incubated at 4° C. for a further 20 minutes, washed a further two times in Perm/Wash buffer and resuspended in 200 μl of MACS buffer for FACS analysis. Intra-nuclear staining for BrdU was carried out using anti-BrdU-APC flow kit (BD Biosciences, Oxford, UK), according to the manufacturer's instructions. Once cells were appropriately stained they were analysed using a LSRII flow cytometer (BD Biosciences) or Fortessa flow cytometer (BD Biosciences) and the data was further analysed using FlowJo software (Tree Star).

### Results

T_{CXCR4} express higher levels of proteins associated with memory differentiation (Bcl2, CD122 and CD62L; FIG. 3). The retention of CD62L expression despite robust proliferation and expansion is a feature associated with self-renewal.

### Example 4

### Materials and Methods

### In Vivo A20 Subcutaneous Tumour Model

B6 mice were used as donors, mice were sacrificed and spleens harvested. Splenic single cell suspensions were sorted using Miltenyi pan T cell sorting beads (130-095-130). One LS column was used per 100×10⁶ cells. T cells were next activated with ConA and IL-7. On day 2 activated T cells were transduced either with CXCR4-IRES-GFP or control viral supernatant. On the same day BALB/c recipient mice were weighed and then irradiated with 4Gy (having being pre-treated with Baytril). On day 3, mice received a second fraction of irradiation (4Gy). Four hours later recipient mice were given 5×10⁶ B6 BM cells intravenously (donor BM cell were depleted for T cells using CD4 (130-049-201) and CD8 (130-049-401) Miltenyi beads (ratio 10 μl/10 μl and 80 μl with MACs buffer per 10⁷ BM cells) and passed through a LD column). After BM administration, 5×10⁶ A20 cells were injected into the right shaved flank of each recipient. On day 5, recipient mice were either injected with T_{CXCR4} or T_{Control} cells at the specified dose.

### In Vivo A20 Intra-Bone Tumour Model

B6 mice were used as donors, mice were sacrificed and spleens harvested. Splenic single cell suspensions were sorted using Miltenyi pan T cell sorting beads (130-095-130). One LS column was used per 100×10⁶ cells. T cells were subsequently activated with ConA and IL-7, as previously. On day 2 activated T cells were transduced either with CXCR4-IRES-GFP or control viral supernatant. On the same day, BALB/c recipient mice were weighed and then irradiated with 4Gy (having being pre-treated with Baytril). On day 3, mice received a second fraction of irradiation (4Gy). Four hours later recipient mice were given 5×10⁶ B6 T-cell depleted BM cells. After BM administration, mice were anaesthetized and 5×10⁵ A20 (HuCD34:luc) cells were injected into the right tibial BM cavity via the tibial plateau. A20 cells were spiked with 5×10⁵ CD45.1 TCD BM as a positive control for injection. On day 5, recipient mice were either injected with T^{CXCR4} or T^{Control} cells at a dose of 0.5-1×10⁵ cells. On day 11-18 after T cell transfer, recipient mice were sacrificed and right and left hind legs harvested. BM was flushed separately from the right and left tibia. Harvested BM was RBC lysed in ACK lysis buffer 2 ml for 2 min. Cell numbers were counted and samples re-suspended ready for FACS analysis. Disease burden was assessed by staining for HuCD34 and CD19, T cell numbers were established by CD4/CD8 staining and GFP positivity. If no malignant cells were detected, confirmation of successful A20 injection was obtained by the presence of CD45.1 BM cells.

### Results

In two models to test anti-tumour functions of adoptively transferred T cells, T^{CXCR4} function better than control cells (FIG. 4). These data indicate the potential clinical relevance of ectopic expression of CXCR4 in therapeutic T cells.

All publications mentioned in the above specification are herein incorporated by reference. Various modifications and variations of the described cells, compositions, uses and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. Use of C-X-C chemokine receptor type 4 (CXCR4) for:
(a) increasing the capacity for self-renewal and/or persistence in a T cell;
(b) increasing the capacity for engraftment in a T cell; and/or
(c) increasing the memory function of a T cell.

2. The use of claim 1, wherein the T cell is genetically engineered to express the CXCR4.

3. The use of claim 2, wherein the T cell is transduced or transfected with a vector comprising a polynucleotide encoding the CXCR4.

4. The use of any preceding claim, wherein the CXCR4:
(a) is encoded by a polynucleotide comprising a nucleotide sequence that has at least 70% identity to SEQ ID NO: 1 or 3; and/or
(b) comprises a protein that has at least 70% identity to SEQ ID NO: 2 or 4.

5. The use of any preceding claim, wherein the T cell has been further genetically engineered to express a T cell receptor (TCR) and/or chimeric antigen receptor (CAR).

6. A method of:
(a) increasing the capacity for self-renewal and/or persistence in a T cell;
(b) increasing the capacity for engraftment in a T cell; and/or
(c) increasing the memory function of a T cell,
wherein the method comprises the step of genetically engineering the T cell to express C-X-C chemokine receptor type 4 (CXCR4).

7. A genetically engineered T cell obtainable through the use of any one of claims 1-5 or by the method of claim 6.

8. A genetically engineered T cell which has an increased capacity for self-renewal and/or persistence; engraftment; and/or memory function.

9. The genetically engineered T cell of claim 8, wherein the T cell has been genetically engineered to express C-X-C chemokine receptor type 4 (CXCR4).

10. The genetically engineered T cell of any one of claims 7-9, wherein the T cell has been further genetically engineered to express a T cell receptor (TCR) and/or chimeric antigen receptor (CAR).

11. A pharmaceutical composition comprising the genetically engineered T cell of any one of claims 7-10 and a pharmaceutically acceptable carrier, diluent or excipient.

12. A genetically engineered T cell according to any one of claims 7-10 for use in therapy.

13. A genetically engineered T cell according to any one of claims 7-10 for use in the treatment of cancer or a viral infection.

14. The genetically engineered T cell for use according to claim 12 or 13, wherein the subject to be treated is not conditioned before administration of the T cell.

15. The genetically engineered T cell for use according to claim 14, wherein the subject to be treated does not undergo chemotherapy or radiotherapy conditioning before administration of the T cell.

16. The genetically engineered T cell for use according to any one of claims 12-15, wherein the T cells are administered in a single dose.

17. A method of engrafting a subject with T cells, comprising the steps:
(a) providing a T cell which has been genetically engineered to express CXCR4; and
(b) administering the T cell provided by step (a) to the subject,
preferably wherein the subject is not conditioned before administration of the T cell.

18. A method of treating or preventing cancer or a viral infection, comprising the steps:
(a) providing a T cell which has been genetically engineered to express CXCR4; and
(b) administering the T cell provided by step (a) to a subject in need thereof,
preferably wherein the subject to be treated is not conditioned before administration of the T cell.

19. The method of claim 17 or 18, wherein the genetically engineered T cell provided by step (a) has been further genetically engineered to express a T cell receptor (TCR) and/or chimeric antigen receptor (CAR).

20. The method of any one of claims 17-19, wherein the subject to be treated does not undergo chemotherapy or radiotherapy conditioning before administration of the T cell.

21. The method of any one of claims 17-20, wherein the T cells are administered in a single dose.
